(19) United States
(12) Patent Application Publication
(10) Pub. No.: US 2007/0135469 A1
(43) Pub. Date: Jun. 14, 2007

(54) Vinylsulfonate and vinylsulfonamide compounds

(76) Inventor: Shiow-Ju Lee, Tainan (TW)

Correspondence Address: FISH & RICHARDSON PC, P.O. BOX 1022, MINNEAPOLIS, MN, 55440-1022, US

(21) Appl. No.: 11/635,825

(22) Filed: Dec. 8, 2006

Related U.S. Application Data
(60) Provisional application No. 60/748,514, filed on Dec. 8, 2005.

(51) Int. Cl. A61K 31/44; A61K 31/40; A61K 31/255; A61K 31/18

(52) U.S. Cl. 514/277; 514/408; 514/602; 514/517

(57) ABSTRACT

A method for activating PPARγ or inhibiting NO production with an effective amount of a vinylsulfonate or vinylsulfonamide compound of the following formula: 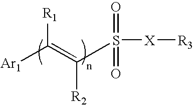
wherein Ar₁ is aryl or heteroaryl, each of R₁, R₂, and R₃, independently, is hydrogen, alkyl, aryl, cyclyl, heteroaryl, or heterocyclyl, X is O or NR, R being hydrogen, alkyl, aryl, cyclyl, heteroaryl, or heterocyclyl, and n is 0, 1, 2, 3, or 4. This invention also covers a method of treating a NO-related disease or PPARγ-related disease with such a vinylsulfonate or vinylsulfonamide compound.

### RELATED APPLICATIONS

This application claims priority to U.S. Provisional Application No. 60/748,514, filed on Dec. 8, 2005, the contents of which are incorporated herein by reference.

### BACKGROUND

Nitric oxide (NO) is an important pleiotropic molecule mediating a wide range of physiological and pathological processes. Overproduction of NO has been implicated in various pathological processes including septic shock, virus infection, tissue damage following inflammation, cancer, atherosclerosis, and arthritis. See, e.g., Alcaraz et al., Current Pharmaceutical Design, 2002: 8, 215.

NO is produced from L-arginine and molecular oxygen by three distinct isoforms of nitric oxide synthase (NOS), i.e., neural NOS (nNOS), endothelial NOS (eNOS), and inducible NOS (iNOS). Among the three NOSs, iNOS can be induced by endotoxins or cytokines (e.g., TNF α and IL-6) to produce a high level of NO. Inhibiting expression or activity of iNOS can reduce NO overproduction.

Peroxisome proliferator-activated receptor γ (PPARγ), a ligand-activated transcription factor, mediates inhibition of iNOS expression. See, e.g., Cernuda-M. Ricote et al., Nature 391, 79. It is also involved in inhibiting on cytokine release and vascular smooth cell growth and migration. PPARγ agonists have been used to protect against or treat many diseases, such as inflammations, atherosclerosis, obesity, and Type II diabetes. See, e.g., Youssef et al., J of Biomed. and Biotech., 2004: 3, 156.

### SUMMARY

This invention is based on a surprising discovery that a number of vinylsulfonate or vinylsulfonamide compounds activate PPARγ and suppress NO production.

Thus, one aspect of this invention is a method for activating PPARγ, which includes administering to a subject in need thereof an effective amount of a vinylsulfonate or vinylsulfonamide compound of the following formula: 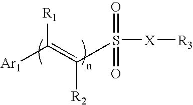
wherein Ar₁ is aryl or heteroaryl, each of R₁, R₂, and R₃, independently, is hydrogen, alkyl, aryl, cyclyl, heteroaryl, or heterocyclyl, X is O or NR, R being hydrogen, alkyl, aryl, cyclyl, heteroaryl, or heterocyclyl, and n is 0, 1, 2, 3, or 4.

In some of the above-described compounds, X can be O or NH, 2. The method of claim 1, wherein X O or N; Ar₁ can be phenyl; R₃ can be phenyl or naphthyl, each of which may optionally substituted with halo, alkyl, alkoxy, aryloxy, carboxy, alkoxycarbonyl, alkylcarbonyl, aldehyde, or aminocarbonyl; n can be 0 or 1.

The term “alkyl” refers to a straight or branched hydrocarbon, containing 1-10 carbon atoms. Examples of alkyl groups include, but are not limited to, methyl, ethyl, n-propyl, i-propyl, n-butyl, i-butyl, and t-butyl.

The term “aryl” refers to a 6-carbon monocyclic, 10-carbon bicyclic, 14-carbon tricyclic aromatic ring system wherein each ring may have 1 to 4 substituents. Examples of aryl groups include, but are not limited to, phenyl, naphthyl, and anthracenyl.

The term “cyclyl” refers to a saturated and partially unsaturated cyclic hydrocarbon group having 3 to 12 carbons. Examples of cyclyl groups include, but are not limited to, cyclopropyl, cyclobutyl, cyclopentyl, cyclopentenyl, cyclohexyl, cyclohexenyl, cycloheptyl, and cyclooctyl.

The term “heteroaryl” refers to an aromatic 5-8 membered monocyclic, 8-12 membered bicyclic, or 11-14 membered tricyclic ring system having one or more heteroatoms (such as O, N, or S). Examples of heteroaryl groups include pyridyl, furyl, imidazolyl, benzimidazolyl, pyrimidinyl, thienyl, quinolinyl, indolyl, and thiazolyl.

The term “heterocyclyl” refers to a nonaromatic 5-8 membered monocyclic, 8-12 membered bicyclic, or 11-14 membered tricyclic ring system having one or more heteroatoms (such as O, N, or S). Examples of heterocyclyl groups include, but are not limited to, piperazinyl, pyrrolidinyl, dioxanyl, morpholinyl, and tetrahydrofuranyl.

Alkyl, aryl, cyclyl, heteroaryl, and heterocyclyl mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cyclyl, heterocyclyl, in which alkyl, alkenyl, alkynyl, alkyloxy, aryl, heteroaryl cyclyl, and heterocyclyl are optionally further substituted.

The compound described above may contain one or more double bonds. Thus, they may occur as cis- or trans-isomeric forms. All such isomeric forms are contemplated.

Table 1 below show exemplary compounds that can be used to practice this invention:

TABLE 1

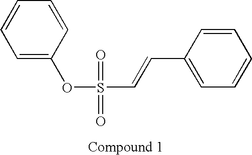

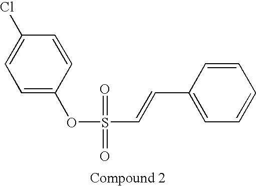

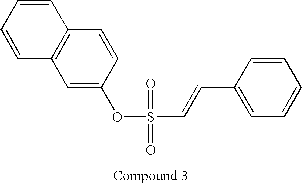

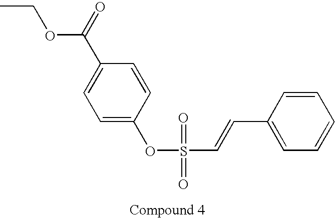

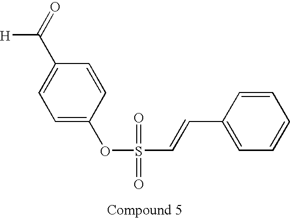

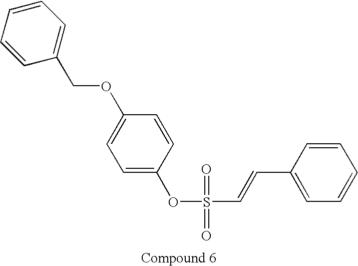

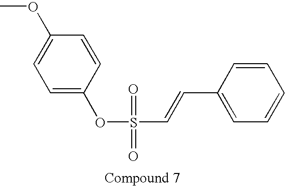

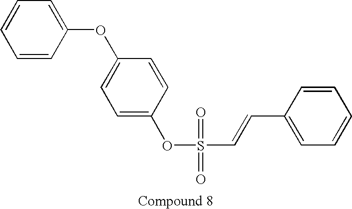

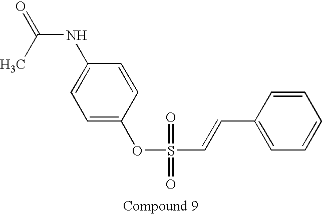

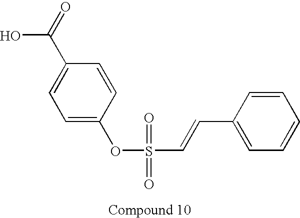

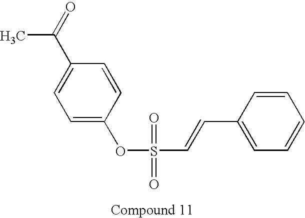

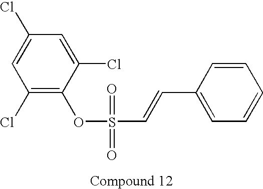

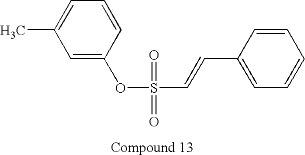

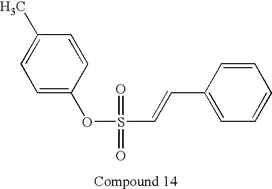

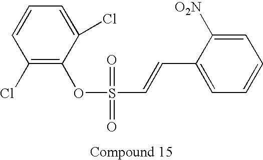

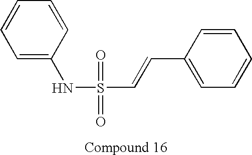

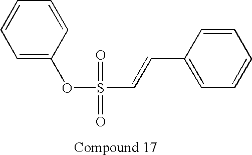

Another aspect of this invention is a method of inhibiting NO production, which includes administering to a subject in need thereof an effective amount of the vinylsulfonate or vinylsulfonamide compound described above.

Still another aspect of this invention is a method of treating a PPARγ-related or NO-related disease, e.g., atherosclerosis, by administering to a subject in need thereof an effective amount of the above-described vinylsulfonate or vinylsulfonamide compound.

Also within the scope of this invention is a composition containing the above-described vinylsulfonate or vinylsulfonamide compound and a pharmaceutically acceptable carrier for use in treating a PPARγ-related or NO-related disease, as well as the use of such a composition for the manufacture of a medicament for treating such a disease.

The details of one or more embodiments of the invention are set forth in the description below. Other features, objects, and advantages of the invention will be apparent from the description and from the claims.

### DETAILED DESCRIPTION

Vinylsulfonate or vinylsulfonamide compounds can be synthesized according to methods known in the art. See, e.g., Rouch et al., J. Am. Chem. Soc., 1998: 120, 10994. Scheme 1 below illustrates a method of synthesizing such compounds.

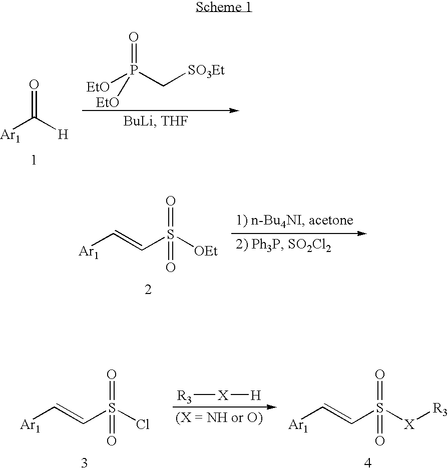

In this scheme, ethyl vinylsulfonate ester 2 is first prepared from aldehyde 1 via Horner-Wadsworth-Emmons reaction. The ester is then hydrolyzed and reacted with SO₂Cl₂ to form sulfonyl chloride 3, which is subsequently coupled with amine or alcohol to provide vinylsulfonate or vinylsulfonamide 4.

The synthetic route shown above may include other transformations or protection and deprotection steps in order to make the desired compounds. Useful synthetic chemistry transformations and protecting group methodologies include, e.g., those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Some of the vinylsulfonate compounds of the formula shown above are commercially available. For examples, compounds 1-17 can be purchased from Chemical Diversity Labs, Inc. (CA, USA).

This invention includes a method of suppressing NO production or activating PPARγ by administering to a subject in need thereof an effective amount of one of the vinylsulfonate or vinylsulfonamide compounds described above. It also includes a method of treating a NO-related disease or PPARγ-related disease with an effective amount of such a compound. The term “an effective amount” refers to the amount of the compound which is required to confer the desired effect in the subject. Effective amounts may vary, as recognized by those skilled in the art, depending on route of administration, excipient usage, and the possibility of co-usage with other agents. The term “treating” refers to administering the one of the above-described vinylsulfonate or vinylsulfonamide compounds to a subject that suffers from a NO-related disease or PPARγ-related disease, or has a symptom of the disease, or has a predisposition toward the disease, with the purpose to cure, heal, alleviate, relieve, alter, remedy, ameliorate, improve, or affect the disease, the symptoms of the disease or the predisposition toward the disease.

A NO-related disease is associated with overproduction of NO. Examples of a nitric oxide-related disorder include, but are not limited to, an inflammatory disease, arthritis, or atherosclerosis. An inflammatory disease is characterized by a local or systemic, acute or chronic inflammation. Examples of an inflammatory diseases include systemic lupus erythematosus, encephalitis, meningitis, hepatitis, sepsis, sarcoidosis, psoriasis, Type I diabetes conjunctivitis, asthma, arteriosclerosis, chronic obstructive pulmonary disease, sinusitis, dermatitis, inflammatory bowel disease, ulcerative colitis, Crohn's disease, Behcet's syndrome, and graft rejection.

A PPARγ-related disease refers to a disease that can be prevented, treated, ameliorated, or cured by activating PPARγ. Examples of a PPARγ-related disorder include, but are not limited to, inflammations, atherosclerosis, cancer, obesity, and Type II diabetes.

To practice the method of the present invention, a composition containing one of the vinylsulfonate or vinylsulfonamide compounds described above and a pharmaceutical acceptable carrier can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol and water. In addition, fixed oils are conventionally employed as a solvent or suspending medium (e.g., synthetic mono- or di-glycerides). Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having an active vinylsulfonate or vinylsulfonamide compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active vinylsulfonate or vinylsulfonamide compound. Examples of other carriers include colloidal silicon oxide, magnesium stearate, cellulose, sodium lauryl sulfate, and D&C Yellow #10.

The vinylsulfonate and vinylsulfonamide compounds described herein can be preliminarily screened by an in vitro assay for one or more of their desired activities, e.g., inhibiting NO production. Compounds that demonstrate high activities in the preliminary screening can further be screened for their efficacy by in vivo assays. For example, a test compound can be tested in a mouse model to assess its effect in treating atherosclerosis. See, e.g., Kauser et al., Am. J. Physiol.: Heart Circ. Physiol. 2000, 278: 1679-1685; and Detmers et al., J. of Immun. 2000, 165: 3430-3435. Based on the results, an appropriate dosage range and administration route can also be determined.

The specific examples below are to be construed as merely illustrative, and not limitative of the remainder of the disclosure in any way whatsoever. Without further elaboration, it is believed that one skilled in the art can, based on the description herein, utilize the present invention to its fullest extent.

### EXAMPLE 1

### Inhibition of Nitric Oxide

RAW 264.7 cells (mouse leukaemic monocyte macrophage cells) were maintained in sodium pyruvate-free DMEM (Hyclone) with 4 mM glutamine, 4500 mg/L glucose, 1% non-essential amino acids (Biological Industries, Israel), and 10% bovine serum (FetaClone III, HyClone).

The cells were seeded at a density of 70,000 cells/well in 96-well culture plates and cultured in an incubator under 5% CO₂ at 37° C. After 24 h, the medium was replaced with one containing stimulus lipopolysaccharides (5 μg/mL, Chemicon International, California)/IFNc (20 ng/mL, R&D systems Inc., Minnesota) and a test compound. After 18 h, nitric oxide production in each supernatant was measured using the Nitrate/Nitrite assay kit (Cayman Chemical). Nitric oxide levels were determined spectrophotometrically with the Griess reagent at OD405. At least five different concentrations of the compound were used. Based on the data thus obtained, IC₅₀ values with respect to nitric oxide production were determined.

Compounds 1-17 were tested in this assay. Unexpectedly, all of them effectively inhibited production of NO stimulated by LPS.

### EXAMPLE 2

### Scintillation Proximity Assay

A bottle of Yttrium silicate beads (Amersham Pharmacia Biotech, catalog No. RPN143) were suspended in 50 ml of an assay buffer containing 10 mM Tris-Cl, pH 7.2, 1 mM EDTA, 10% (w/v) glycerol, 10 mM sodium molybdate, 1 mM dithiothreitol, 0.5 mM phenylmethylsulfonyl fluoride, 2 μg/ml benzamidine, and 0.01% sodium azide. The suspension was diluted 5× with a solution containing 10 mM Tris-Cl, pH 7.2, 1 mM EDTA, 10% (w/v) glycerol, 10 mM sodium molybdate, 1 mM dithiothreitol, 0.5 mM phenylmethylsulfonyl fluoride, 2 μg/ml benzamidine, 0.1% dry milk powder, 10 nM ³H-BRL49653 (American Radiolabeled Chemicals, Inc.), 5 nM GST-PPARγ (LBD) recombinant protein, goat anti-GST Ab (Amersham Pharmacia Biotech, diluted 200×), and a test compound. Binding competition between ³H-BRL49653 and each compound to PPARγ was carried out overnight at 10° C. with shaking. Radioactivity was quantified in a TopCount.NXT™ microplate scintillation and liminscence counter (Packard Inc.). Each compound was tested at 8 or more different concentrations. The IC₅₀ values with respect to inhibiting ³H-BRL49653's binding with PPARγ were then calculated by a known method (Elbrecht, A. et al., J Biol Chem, 1999: 274, 7913-7922 and Nichols, J. et al., Anal. Biochem. 1998: 257, 112-119).

Compounds 1-17 were tested in this assay. The results show that all of them, unexpectedly, bound PPARγ in competition with ³H-BRL49653, some of them having IC₅₀ values lower than 1 μM in inhibiting ³H-BRL49653's binding with PPARγ.

### EXAMPLE 3

### PPARγ Trans-Activation Assay

PPARγ LBD was fused with Gal4 DBD (residues 1-147) to generate a chimeric construct Gal4^{DBD}-PPARγ^{LBD} in a pSG424 fusion vector. CV1 cells (African green monkey fibroblast cells), seeded in Dulbecco's Modified Eagle Medium containing 10% fetal calf serum at 2.5×10⁴ cells/well in 24-well plates, were transfected with plasmids of chimeric receptor pSG424-PPARγ^{LBD}, UAS₃ luciferase reporter gene (see Chawla, A. et al., Proc. Nat. Aca. Sci. U.S.A., 2003: 1268), and pCMV-βGal using FuGene⁶ (Roche). The transfected cells were washed twice with culture medium and then a test compound was added. After 18-24 h, luciferase and β-galactosidase assays were conducted using the Steady-Glo luciferase assay system (Promega) and the Galacto-Star assay system (Tropix), respectively. Luminescence was measured in a TopCount.NXT™ microplate scinetillation and luminescence counter (Packard, Inc.). The luciferase activity was normalized with the β-galactosidase activity. The PPARγ trans-activity stimulated by 15-deoxy-delta12,14-prostglandin J2 (10 μM) was used as control.

Compounds 1-17 were tested. The results show that all of them unexpectedly promoted PPARγ activity.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to above-described vinylsulfonate or vinylsulfonamide compounds also can be made, screened for the above-described activities and used to practice this invention. Thus, other embodiments are also within the claims.

What is claimed is:

1. A method for activating PPARγ, comprising administering to a subject in need thereof an effective amount of a compound of the following formula: 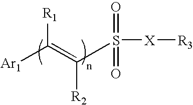
wherein
Ar₁ is aryl or heteroaryl;
each of R₁, R₂, and R₃, independently, is hydrogen, alkyl, aryl, cyclyl, heteroaryl, or heterocyclyl;
X is O or NR, R being hydrogen, alkyl, aryl, cyclyl, heteroaryl, or heterocyclyl; and
n is 0, 1, 2, 3, or 4.

2. The method of claim 1, wherein X O or N.

3. The method of claim 2, wherein Ar₁ is phenyl.

4. The method of claim 3, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

5. The method of claim 3, wherein n is 1.

6. The method of claim 5, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

7. The method of claim 1, wherein Ar₁ is phenyl.

8. The method of claim 1, wherein n is 1.

9. The method of claim 1, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

10. The method of claim 1, wherein the compound is selected from Table 1.

11. A method for inhibiting NO production, comprising administering to a subject in need thereof an effective amount of a compound of the following formula: 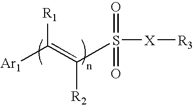
wherein
Ar₁ is aryl or heteroaryl;
each of R₁, R₂, and R₃, independently, is hydrogen, alkyl, aryl, cyclyl, heteroaryl, or heterocyclyl;
X is O or NR, R being hydrogen, alkyl, aryl, cyclyl, heteroaryl, or heterocyclyl; and
n is 0, 1, 2, 3, or 4.

12. The method of claim 11, wherein X O or N.

13. The method of claim 12, wherein Ar₁ is phenyl.

14. The method of claim 13, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

15. The method of claim 13, wherein n is 1.

16. The method of claim 15, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

17. The method of claim 11, wherein Ar₁ is phenyl.

18. The method of claim 11, wherein n is 1.

19. The method of claim 11, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

20. The method of claim 11, wherein the compound is selected from Table 1.

21. A method for treating atherosclerosis, comprising administering to a subject in need thereof an effective amount of a compound of the following formula: 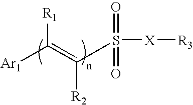
wherein
Ar₁ is aryl or heteroaryl;
each of R₁, R₂, and R₃, independently, is hydrogen, alkyl, aryl, cyclyl, heteroaryl, or heterocyclyl;
X is O or NR, R being hydrogen, alkyl, aryl, cyclyl, heteroaryl, or heterocyclyl; and
n is 0, 1, 2, 3, or 4.

22. The method of claim 21, wherein X O or N.

23. The method of claim 22, wherein Ar₁ is phenyl.

24. The method of claim 23, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

25. The method of claim 23, wherein n is 1.

26. The method of claim 25, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

27. The method of claim 21, wherein Ar₁ is phenyl.

28. The method of claim 21, wherein n is 1.

29. The method of claim 21, wherein R₃ is phenyl or naphthyl, each of which may optionally substituted with halo, alkyl, alkoxy, aryloxy, carboxy, alkoxycarbonyl, alkylcarbonyl, aldehyde, or aminocarbonyl.

30. The method of claim 21, wherein the compound is selected from Table 1.
